(19) United States
(12) Patent Application Publication
(10) Pub. No.: US 2014/0276356 A1
(43) Pub. Date: Sep. 18, 2014

(54) Novel Enhanced Device and Composition for Local Drug Delivery

(71) Applicant: Teleflex Medical Incorporated, Research Triangle Park, NC (US)

(72) Inventors: John C. Victor, Kunkletown, PA (US); David T. Rowe, Sinking Spring, PA (US); Jeffrey Vitullo, Pottstown, PA (US)

(73) Assignee: Teleflex Medical Incorporated, Research Triangle Park, NC (US)

(21) Appl. No.: 14/212,622

(22) Filed: Mar. 14, 2014

Related U.S. Application Data
(60) Provisional application No. 61/781,831, filed on Mar. 14, 2013.

(51) Int. Cl. A61L 31/16; A61L 31/00; A61N 5/06

(52) U.S. Cl. CPC A61L 31/16; A61N 5/062; A61L 31/005
USPC 604/20; 427/2.25

(57) ABSTRACT

A medical device for delivering a reagent, such as a pharmaceutical agent, a diagnostic agent, a nutrient, or another type of reagent, to an intravascular or intralumenal location, is disclosed. The medical device has a coating, where exposing the coating to light severs a photosensitive bond that releases the reagent into the immediate vicinity of the location.

### CROSS-REFERENCE TO RELATED APPLICATIONS

This application claims priority to U.S. Provisional Application No. 61/781,831, entitled “NOVEL ENHANCED DEVICE AND COMPOSITION FOR LOCAL DRUG DELIVERY,” filed Mar. 14, 2013, the disclosure of which is hereby incorporated by reference in its entirety.

### FIELD OF THE DISCLOSURE

The present disclosure relates a medical devices used in the treatment of cardiovascular disorders, such as atherosclerosis and restenosis. The disclosure relates to coated medical devices that provide a photocleavable coating, where light provokes the release of a pharmaceutical agent.

### BACKGROUND OF THE DISCLOSURE

Many disorders are treated with pharmaceuticals. For example, high blood cholesterol is treated with atorvastatin, which in inhibits an enzyme in the cholesterol biosynthetic pathway. Omeprazole, which inhibits a proton pump, is a drug used to treat stomach ulcers. Furosemide, which acts on an ion transporter, is used to treat hypertension. Most pharmaceuticals, including the above, are administered systemically, for example, orally, by injection, or by infusion. Efficacy and safety of some pharmaceuticals can be improved, or made possible, by local administration. For example, where a medical device such as a stent or an angioplasty balloon is implanted into a blood vessel, the medical device can be configured for controlled, local release of a pharmaceutical agent in the region of the implanted device.

In the context of cardiovascular diseases, drug-eluting stents and balloons have been used for preventing or treating adverse events, such as thrombosis (pathological blood clot), restenosis, neoatherosclerosis, myocardial infarction, and mortality (see, e.g., Tepe et al (2010) Cardiovasc. Surg. 51:125-143; Indermuehle et al (2013) Heart. 99:327-333; Otsuka et al (2012) Thrombosis. Article ID 608593 (16 pages)). Restenosis, which can occur after surgical treatment of a narrowed vascular lumen, is the narrowing of vascular lumen by way of pathological growth of endothelial cells of the vessel wall. A related set of problems is that the medical device itself can produce adverse events, such as inflammation, thrombosis, and delayed healing (see, e.g., Waksman et al (2009) Circ. Cardiovasc. Intervcent. 2:352-358; Cutlip (2011) Circulation. 123:2779-2781). Adverse events in cardiovascular disease also take the form of laboratory data, such as abnormal Q waves of an electrocardiogram. Moreover, difficulties in treatment can arise where cardiovascular lesions are long, where the vessels are small-diameter blood vessels, with saphenous vein grafts, and where the patient is in a high-risk category, such as diabetes or renal failure (see, e.g., Maluenda et al (2012) Circ. Cardiovasc. Interv. 5:12-19). Pharmaceutical agents that have been administered by way of drug-eluting medical devices include anti-proliferative agents and anti-inflammatory agents. Anti-proliferative agents, such as those used to treat neoplastic diseases, are classed as those that are cytostatic and those that are cytotoxic. The present disclosure meets the unmet need of treating or preventing conditions that can benefit by local drug administration, such as cardiovascular disease, by way of device that includes a photocleavable pharmaceutical agent.

### SUMMARY OF THE DISCLOSURE

Briefly stated, the disclosure provides a medical device for delivering a reagent, such as a medicament, a pharmaceutical agent, a diagnostic agent, a nutrient, a biological such as an antibody, or another type of reagent, to an intravascular or intralumenal location, is disclosed. The medical device has a coating, where exposing the coating to light severs a photosensitive bond that releases the reagent into the immediate vicinity of the location. The lumen can be intravascular, lymphatic, it can be a duct such as the bile duct, it can be a tract such as part of the urinary tract. The intraluminal location can be an atherosclerotic lesion, without implying any limitation.

The disclosure also provides a medical device configured for delivering at least one reagent to an intraluminal location, or other location in the body, the medical device comprising a surface and a coating, wherein the coating is modified by a photosensitive linker that covalently binds the at least one pharmaceutical agent, the photosensitive linker comprising: (a) a first functional group that covalently binds the pharmaceutical agent; (b) a photosensitive moiety that is adapted to cleaved by exposure to light; and (c) a second functional group that maintains contact with the coating; wherein the coating on the medical device is configured to deliver and, with exposure to light, release the at least one reagent in an effective amount that contacts the location.

Location can be intravascular, intracardial, intraocular, a lumen of the urinary tract, a duct such as bile duct, a lymphatic tract, a lumen of the digestive tract, and so on.

Also provided is the above medical device, wherein the reagent is a pharmaceutical agent, a diagnostic agent, a medicament, or a nutrient. Also provided is above medical device, wherein the device is configured for delivering at least one medicament to an intraluminal location that comprises atherosclerotic plaque or a location at risk for restenosis. Also encompassed is above medical device, wherein the device is configured for delivering at least pharmaceutical agent to an intraluminal location that comprises atherosclerotic plaque or a location at risk for restenosis.

Also embraced is above medical device, wherein the location is tissue that is at risk for restenosis, and wherein the location had been treated for atherosclerotic plaque. Also contemplated is above medical device, wherein the device is configured for delivering at least one medicament to an intraluminal location that comprises a blood clot or embolism. In another aspect, what is provided is above medical device that comprises at least one angioplasty balloon, a stent, and a vascular device, that is configured for temporary or permanent placement.

Moreover, in another aspect, what is provided is above medical device, wherein the second functional group maintains contact with the coating by hydrogen bonds, and not by one or more covalent bonds. What is also provided is above medical device, wherein the second functional group maintains contact with the coating by at least one covalent bond. Also provided is above medical device, wherein the at least one reagent is cytostatic. Also provided is above medical device, wherein the at least one reagent is cytotoxic.

Furthermore, what is provided is above medical device, where the at least one reagent includes a taxol, or an analogue thereof. Also provided is above medical device, wherein the at least one pharmaceutical agent is paclitaxel. In another aspect, what is provided is above medical device, wherein the coating comprises at least one of a polyurethane or a polysiloxane.

Also provided is above medical device, wherein the photosensitive linker comprises one or both of, at least one double bond that links two carbon atoms, at least one azo linkage, or at least one peroxide linkage. Also provided is above medical device,further comprising a photo up-conversion material. Moreover, what is further embraced is above medical device, further comprising a photo up-conversion material that comprises inorganic phosphor crystals. Also provided is above medical device, further comprising a light-emitting diode that delivers a photocleavably effective light to the coating.

Moreover, what is provided is above medical device, further comprising a second medical device having at least one optical fiber that delivers a photocleavably effective light to the coating. Moreover, what is provided is above medical device, that occurs as an integral unit with the second medical device. In kit embodiments, what is provided is a kit comprising the above medical device, and a second medical device having at least one optical fiber that delivers a photocleavably effective light to the coating.

Also provided is a kit comprising the above medical device, wherein the medical device comprises an angioplasty balloon that is coated with at least one photocleavable reagent, and a second medical device having at least one optical fiber that delivers a photocleavably effective light to the coating.

In methods embodiments, what is provided is a method for delivering (or releasing) at least one reagent to an intraluminal location, or other location in the body, comprising positioning the above medical device in said intraluminal location, or other location in the body, and irradiating the coating with a cleavably effective amount of radiation. Also provided is above method, wherein the intraluminal location is an atherosclerotic lesion, or a lesion at risk for restinosis, and wherein the coating comprises a photocleavable link that is linked to paclitaxel. In manufacturing embodiments, what is provided is a method for manufacturing the above medical device, in each of the above-disclosed embodiments, comprising attaching a photocleavable linkage and a medicament to a surface of a non-coated medical device, wherein the photocleavable linkage tethers the medicament to the surface of the medical device.

In yet another methods embodiment, what is provided is above method, wherein the intraluminal location is an atherosclerotic lesion, or a location that is at risk for restenosis, and the reagent is an anti-proliferative agent.

The present disclosure encompasses all possible combinations of the above embodiments, and encompasses all possible disclosures of each independent claim with its dependent claims. For example, what is encompassed is an invention that is the combination of: Claim 1+claim 2; or the combination of: Claim 1+claim 2+claim 3; or the combination of claim 1+claim 3+claim 4; or the combination of claim 1+claim 2+claim 3+claim 4; and the like.

### BRIEF DESCRIPTION OF THE FIGURES

FIG. 1 is a schematic representation of an example of a suitable medical device configured for delivering at least one reagent to an intraluminal location, or other location in the body according to an embodiment.

### DETAILED DESCRIPTION OF THE DISCLOSURE

As used herein, including the appended claims, the singular forms of words such as “a,” “an,” and “the” include their corresponding plural references unless the context clearly dictates otherwise. All references cited herein are incorporated by reference to the same extent as if each individual publication, patent, and published patent application, as well as figures and drawings in said publications and patent documents, was specifically and individually indicated to be incorporated by reference.

### Lightwave Embodiments

Specific wavelengths, or range of wavelengths, and fluence values for photocleaving, for effecting photocleavage are available. What is encompassed is, light in the visible spectrum (390-700 nm). What is encompassed is light of wavelength range of 200-220 nm, 220-240 nm, 240-260 nm, 260-280 nm, 280-300 nm, 300-320 nm, 320-340 nm, 340-360 nm, 360-380 nm, 380-400 nm, 400-420 nm, 420-440 nm, 440-460 nm, 460-480 nm, 480-500 nm, 500-520 nm, 520-540 nm, 540-560 nm, 560-580 nm, 580-600 nm, 600-620 nm, 620-640 nm, 640-660 nm, 660-680 nm, 680-700 nm, 700-720 nm, 720-740 nm, 740-760 nm, 760-780 nm, 780-800 nm, and the like. Also provided is any combination of the above ranges, to provide a broader range, such as 380-460 nm. Also provided are 5 nm increments, and 10 nm increments, in the above ranges. Moreover, what is provides is a photocleavable chemical moiety that is specifically cleaved by one of the above ranges, or optimally cleaved by a combination of two or more of the above ranges, including where the two ranges are separated by 10 or more nanometers. The skilled artisan understands that some dyes have absorption spectra that possess two or more peaks. Also provided is a method of use, comprising using a light emitting device that emits one or more of the above ranges, directing the light to medical device that is coated with photosensitive linker, resulting in partial of full cleavage.

FIG. 1 is a schematic representation of an example of a suitable medical device 10 configured for delivering at least one reagent to an intraluminal location, or other location in the body. The medical device 10 includes a surface 12 and a coating 14 which covers a portion of the surface 12, such as, in an embodiment, the distal tip, and, in another embodiment, a portion of the sidewalls. The coating 14 is modified by a photosensitive linker that covalently binds the at least one reagent agent. The photosensitive linker includes: (a) a first functional group that covalently binds the pharmaceutical agent; (b) a photosensitive moiety that is adapted to cleaved by exposure to light; and (c) a second functional group that maintains contact with the coating 14. The coating 14 on the medical device 10 is configured to deliver and, with exposure to light, release the at least one reagent in an effective amount that contacts the location. Also shown in FIG. 1, this or other embodiments further include a kit 20 for delivering the reagent. In this kit 20 a second device 22 is provided having at least one optical fiber 24 that delivers a photocleavably effective light generated by a light source 26 to the coating 14. The light source is configured to generate any suitable wavelength of light. Examples of suitable wavelengths of light includes light that is substantially ultraviolet, violet, blue, green, yellow, orange, red, infrared, or any combination of two or more of these, for example as provided by separate optical cables, or as provided at different times.

Fluence embodiments that are encompassed include, without limitation, 0.01-0.02 mW/cm², 0.02-0.05 mW/cm², 0.05-0.1 mW/cm², 0.1-0.2 uW/cm², 0.2-0.5 uW/cm², 0.5-1.0 uW/cm², 1.0-2.0 uW/cm², 2.0-5.0 uW/cm², 5.0-10 uW/cm², 10-20 uW/cm², 20-50 uW/cm², 50-100 uW/cm², 100-200 uW/cm², 200-500 uW/cm², 500-1000 uW/cm², 1-2 mW/cm², 2-5 mW/cm², 5-10 mW/cm², 10-20 mW/cm², 20-50 mW/cm², 50-100 mW/cm², 100-200 mW/cm², 200-500 mW/cm², 500-1000 mW/cm², and so on, and any combination thereof. What is provided is one fluence for one wavelength, and a different fluence for a different wavelength of light.

Light can be delivered continuously, or as pulsation. Pulsating light can alternate with two or more different wavelengths of light, or two or more different fluences. Duration of continuous light, as well as pulsation times, include, about 0.01 msec, about 0.02 msec, about 0.05 msec, about 0.1 msec, about 0.2 msec, about 0.5 msec, about 1.0 msec, about 2 msec, about 5 msec, about 10 msec, about 20 msec, about 50 msec, about 100 msec, about 200 msec, about 500 msec, about 1 sec, about 2 sec, about 5 sec, about 10 sec, about 20 sec, about 50 sec, about 100 sec, about 1 min, about 2 min, about 10 min, about 20 min, about 40 min, about 60 min, about 2 h, about 4 h, and so on. The term about can mean plus or minus ten percent, or the term about can mean somewhere in the range between the previous value and the succeeding value of time. These times can indicate the total duration of a continuous light, or they can indicate the total duration of a series of pulses, or they can indicate the time of a single pulse.

### Photocleavable Groups

The present disclosure encompasses, and is not limited to, the following types of photocleavable groups with the following characteristics. In embodiments, the present disclosure provides a photocleavable group, where there is less than ten side products, less than nine, less than eight, less than seven, less than six, less than five, less than five, less than four, less than three, less than two, or zero side products. For example, where a photocleavable group is attached to a polymer, and where the photocleavable group includes a double bond, a side produce can be an aldehyde moiety that is released from the polymer. In a preferred embodiment, separation of the substrate from the protecting group should occur via a primary photochemical process. In another preferred embodiment, the group should be removable with a wavelength that is not absorbed by other components of coated medical device, or components of the physiological milieu. Also preferred, is an excitation wavelength that is greater than 250 nm, and more preferably greater than 300 nm, to minimize absorption by and damage to biological tissue. In other preferred non-limiting embodiments, protecting group is stable in the absence of light. Moreover, what is preferred is that byproducts do not interfere with the photochemical reaction, and are preferably are transparent at the irradiation wavelength, in order to minimize efficacy of the activating light.

Isosbestic point determination can be used to assess the production of side-products. The existence of discrete isosbestic points indicates lack of side products. Side-products can also be determined by high pressure liquid chromatography (HPLC), nuclear magnetic resonance (NMR), mass spectrometry, and the like.

Non-limiting protecting groups include one or more of, for example, alpha-substituted acetophenone; 3′-5′-dimethoxybenzoin, benzyl group; cinnamate ester; coumaryl-methyl-diethyl phosphate; ortho-nitrobenzyl ester, and analogues thereof. Deprotection of ortho-nitrobenzyl groups can be at, for example, 365 nm. Where the source of light provides a range of wavelengths, with a peak at 365 nm, the range can be 360-370 nm, 355-375 nm, 350-380 nm, 345-385 nm, 340-390 nm, and the like, where the starting and end points refer to wavelengths where the brightness of light is 10% that at the wavelength of maximal light. The skilled artisan can derive similar ranges for any given wavelength maximum. Deprotection of polycyclic aromatic hydrocarbons (aqmoc; mcmoc; phmoc) can be at 350 nm, or with a light source that provides, for example, 345-355 nm, 340-360 nm, 335-365 nm, 330-370 nm, and so on. Deprotection of involving cis-trans isomerization, for coumarin (366 nm), vinylic phenols (254 nm), vinylic napthols (350 nm), are provided, where the wavelength that results in maximal deprotection is shown. Sisyl group deprotection can be with light at 204 nm and 254 nm. N-methyl-N-(o-nitro) carbamate deprotection can be with light at 254 nm. 2-Benzylbenzoic acid group deprotection can be with light at 300-390 nm.

3,5-Dimethoxybenzoin (3,5-DMB) derivatives are provided. Molecules with a functional group that is a carboxylic acid can be protected by reaction with 3,5-DMB to give ester. Molecules with a functional group that is a secondary amine can be protected by reaction with 3,5-DMB to give a carbamate.

### Introducing Functional Groups on Polymer Substrates

Treatment with plasma or corona (high voltage which ionizes air or gas) increases the surface tension of plastic medical products by breaking chemical bonds which disrupts the surface. The new bond that is formed can be stronger than just mechanical bonding. Corona treatment optimizes the adhesion properties on polymer-based materials (Medical Systems for Industry, Huppauge, N.Y.). When a plastic substance is placed under the corona discharge, the electrons generated in the corona discharge impact on the treatment surface with energies two to three times that necessary to break the molecular bonds on the surface of most substrates. The resulting free radicals react rapidly with the oxidizing products of the corona discharge, or with adjoining free radicals on the same or different chain, resulting in a cross-link. Oxidation of the solid surface increases the surface tension energy, allowing for better wetting by liquids and promoting adhesion (3DT, Germantown, Wis.).

A plasma is a partially ionized gas generated by applying an electrical field to a gas under partial vacuum. Inert gas plasmas, such as argon and helium, modify surfaces by cross-linking, chain scission, chain branching, and surface roughening. Reactive gas plasmas involving gases such as oxygen, nitrogen, hydrogen, ammonia and hydrogen sulfide, have been shown to introduce new functional groups onto the polymer surface (Gray et al (2003) Applied Surface Science. 217:210-222). Where new groups are introduced, the process has been called, “plasma grafting.” Plasma treatment can introduce cross-linking, where crosslinking density may be to a depth of a few thousand Angstroms. The result is an increase in surface hardness. In the case of silicone rubber, the result of inert gas plasma treatment is a hard skin on the surface (I.-H. Loh (1997 or later) Plasma Surface Modification in Biomedical Products in AST Technical Journal, AST Products, Billerica, Mass.). Gray et al, supra, describe plasma modification of polyurethane. Regarding another polymer, polystyrene, it is the case that this polymer has a hydrophobic, non-wettable surface. Plasma treatment in the presence of oxygen gas alters the surface chemistry to give a hydrophilic surface, where the polystyrene acquires the following functional groups: ether; alcohol; ketone; aldehyde; ester; acid; carbonate (Plasmatech, Inc., Erlanger, Ky.). Plasma treatment with ammonia gas results in amino functional groups. This procedure is effective with polymers such as polystyrene, polyethylene, polypropylene, polydimethylosiloxane, polyvinylidene fluoride, and others (Bryjak et al (2002) European Polymer J. 38:717-726). The present disclosure provides compositions and methods for introducing one or more of the above-disclosed functional groups, for example, by way of corona or plasma methods, into polyurethane, silicone, polysiloxane, polyvinylchloride, polypropylene, polystyrene, block polymers, rake polymers, copolymers, Tecothane®, Tecoflex®, and other polymers and polymeric compositions, without implying any limitation.

### Agents

Agents for delivery or placement, and release, by way of light-induced cleavage of a linker, include pharmaceutical agents, anti-proliferative agents, anti-restenosis agent, medicaments, diagnostics including labeled diagnostics, nutrients, and the like. Agents that can prevent or inhibit proliferation include taxols, such as paclitaxel, topomerase inhibitors, DNA cross-linking agents, DNA damaging agents, and agents that inhibit enzymes that mediate nucleic acid metabolism. Anti-proliferative agents also include anti-viral agents and anti-bacterial agents. The present disclosure provides medical device with a coating that has a photocleavable linker that holds one or more of, paclitaxel, celecoxib, sirolimus, everolimus, zotarolimus, any other type of limus, and analogues thereof.

Also provided are agents that are therapeutic antibodies, such as antibodies that specifically recognize PD-L1, or antibodies that recognize folate receptor (see, e.g., Golay et al (2012) Arch. Biochem. Biophys. 526:146-153; Brahmer et al (2012) New Engl. J. med. 366:2455-2465; Besse et al (2013) Ann. Oncol. 24:90-96). Toxic antibodies include those that specifically bind to a target cell of interest, or to a target lesion, and deliver a toxic compound that kills the cell, or cells in the lesion. Antibodies can be used to deliver a toxic agent or poison, as well as to deliver a protein, such as a cytokine such as interleukin-12, that inhibits or kills proliferative cells, or that potentiates inhibition or killing by another agent (see, e.g., Pasche et al (2012) Clin. Cancer Res. 18:4092-4103). The present disclosure encompasses the delivery of nanoparticles for diagnostic or treatment purposes, and the delivery of encapsulated drugs (see, e.g., Cohen et al (2012) J. Nanobiotechnology. 10:36; Taylor et al (2012) Int. J. Nanomedicine. 7:4341-4352).

Medical device can be used to deliver a cytostatic agent, or a cytotoxic agent, where the agent is immobilized to the device with a photocleavable linker. Anti-proliferative agents can be classed as those that are cytostatic and those that are cytotoxic (see, e.g., Brody, T. (2012) Clinical Trials. Elsevier, New York, N.Y., p. 200-210, 242-243).

A composition that is “labeled” is detectable, either directly or indirectly, by spectroscopic, photochemical, biochemical, immunochemical, isotopic, or chemical methods. For example, useful labels include ³²P, ³³P, ³⁵S, ¹⁴C, ³H, ¹²⁵I, stable isotopes, epitope tags fluorescent dyes, electron-dense reagents, substrates, or enzymes, e.g., as used in enzyme-linked immunoassays, or fluorettes (see, e.g., Rozinov and Nolan (1998) Chem. Biol. 5:713-728).

Manufacturing embodiments included, such as a method for manufacturing comprising applying a coating to a stent, balloon, probe, catheter, and so on. Another manufacturing embodiment is attaching photocleavable linker to the coating, where attaching can be covalent, or entirely by way of non-covalent bonds. Another manufacturing is preparing a slurry, liquid, composite, paste, and the like that comprises a coating material and photocleavable linker, where the method of manufacture necessarily involves the simultaneous application of both coating and photocleavable linker. In some manufacturing embodiments, a reagent (e.g., pharmaceutical; medicament; diagnostic agent) is attached to linker after linker is already bound to the applied coating. In other embodiments, a reagent (e.g., pharmaceutical; medicament; diagnostic agent) is attached to the linker before the linker is attached to the coating. The coating can be a composition of matter that is applied to medical device. Alternatively, the coating can take the form of functional groups that are created at the surface of a polymer by way of corona or plasma techniques, for example, functional groups that are amino groups or aldehyde groups (in this case, coating can be an integral part of medical device).

### Locations in Body

Where medical device of the disclosure is to be used at a specific location in the body, the pathway taken for placing medical device, as well as the location of use, can dictate the configuration (shape and size) and composition of medical device. For example, if medical device needs to be placed in a narrow lumen, the medical device may need to be narrow, or if medical device needs to be placed in a vascular location, medical device may need a special coating that prevents the generation pathological blood clots. Guidance for governing the configuration of medical device, as well as guidance for placing the medical device during actual use, can be provided, for example, by ultrasound or optical coherence tomography (Muraoka et al (2012) 28:1635-1641; Kang et al (2011) Circ. Cardiovasc. Interv. 4:139-145; Alfonso et al (2012) 103:441-464). Medical device can be configured, to provide non-limiting examples, for placement at or near neointimal formation, location at risk for restenosis, atherosclerotic plaque, bile tract, urinary tract, lymphatic duct, intestines, pulmonary tract, and the like. Identification of lesions at risk for restenosis, and identification of patients at risk for restenosis, can be made by available methods (see, e.g., Montalescot et al (1995) Circulation. 92:31-38; Killip et al (1995) J. Nuclear Med. 36:1553-1560; Garg et al (2008) J. Am. College Cardiol. 51:1844-1853). The above methods can be used, for example, for the goal of determining optimal surface density for manufacturing medical device of the present disclosure. Agent can be immobilized, by way of linker, so that release of all linked agent that resides in a square centimeter of medical device surface area (including reagent that resides slightly below the surface, for example, residing within a polymer matrix), results in release of the linked agent into a theoretical volume of 10 mL (a test volume of 10 mL), to give a concentration in the 10 mL of, at least 1 picomolar (pM), at least 10 pM, at least 100 pM, at least 500 pM, at least 1 nanomolar (nM), at least 10 nM, at least 50 nM, at least 100 nM, at least 500 nM, at least 1 micromolar (uM), at least 10 uM, at least 50 uM, at least 100 uM, at least 500 uM, at least 1 millimolar (mM), at least 10 mM, at least 50 mM, at least 100 mM, and the like.

### Proximal and Distal

In the context of a medical device, such as an assembly having a longitudinal aspect, as an assembly of a sheath and dilator, “proximal” refers generally to the end of the assembly that is closest to the physician while “distal” refers generally to the end that is inserted into the patient. Where the terms “proximal-to-distal movement” or “proximal-to-distal force” are used, these terms can refer to the context where the device is being used with the patient, and also in an abstract context, where a physician and patient are not present.

### Coating and Impregnating Medical Device

What is embraced is a formulation for applying to a surface of a medical device, for example, by soaking, where the formulation comprises a dissolved plastic polymer. The dissolved plastic polymer can be more or more of, or any combination of, polyurethane, polyethylene, polyethlyene teraphthalate, ethylene vinyl acetate, silicone, tetrafluoroethylene, polypropylene, polyethylene oxide, polyacrylate, and so on. What is encompassed are coatings, coating solutions, and medical devices that are coated with coating solutions, using Carbothane® family of polycarbonate-based aliphatic and aromatic polyurethanes, Estane®, which is a thermoplastic polyurethane, Pellethane®, which is a family of medical-grade polyurethane elastomers and exceptionally smooth surfaces, Tecoflex®, which is a family of aliphatic polyether polyurethanes, where low durometer versions are particularly suitable for long-term implant applications, Tecothane®, an aromatic polyurethane, Texin®, an aromatic polyether-based polyurethane which allows for very thin gauges (Microspec Corp., Peterborough, N.H.; Lubrizol, Inc., Wickliffe, Ohio; Entec Polymers, Orlando, Fla.). See, U.S. Pat. No. 6,565,591 of Brady, U.S. Pat. No. 7,029,467 of Currier, and U.S. Pat. No. 7,892,469 of Lim, which are hereby incorporated by reference in their entirety. In embodiments, the present disclosure provides the recited polymers for use in coating solutions, or for use in manufacturing the medical device that is to be coated. A reagent, such as an anti-microbial agent, can be bulk distributed in the medical device, for example, by adding to a melted polymer or by soaking until even distribution has occurred.

Alternatively, the medical device can be impregnated or coated with the agent. In embodiments, the disclosure encompasses methods for bulk distribution, gradient distribution, and limited surface distribution. Methods for manufacturing medical devices where an agent is bulk distributed, gradient distributed, or limited surface distributed, are available (see, e.g., U.S. Pat. No. 4,925,668 issued to Khan, et al, U.S. Pat. No. 5,165,952 issued to Solomon and Byron, and U.S. Pat. No. 5,707,366 issued to Solomon and Byron, all of which are incorporated herein by reference).

Coating and impregnation are distinguished. Generally, coating resides on, or adheres to, the exterior surface of medical device. Coating thickness can be, without limitation, about 10 nanometers (nm), about 50 nm, about 100 nm, about 500 nm, about 1.0 micrometers (um), about 10 um, about 50 um, about 100 um, about 500 um, about 1 millimeters (mm), about 5 mm, and so on. Material used for coating can extend into the medical device, and this aspect of the coating can be referred to as an impregnation. Impregnation can extend throughout entire medical device, and where extension throughout device is substantially uniform, the impregnation is a bulk distribution. Impregnation can extend, without limitation, about 10 nanometers (nm), about 50 nm, about 100 nm, about 500 nm, about 1.0 micrometers (um), about 10 um, about 50 um, about 100 um, about 500 um, about 1 millimeters (mm), about 5 mm, and so on, from the surface into medical device. Alternatively, device can be manufactured so that the an agent does not reside on the surface, but resides only in interior of medical device. Use of the term “coating” or “impregnation” can depend on whether the coating or the impregnation is functionally more important.

The disclosed polymers can be used for manufacturing a medical device itself, as well as for coating the manufactured medical device and for impregnating the manufactured medical device.

### French Size

Diameters of catheters, cannulas, tubes, and such, can be labeled by French size. The disclosure provides a tube with a French size that is, to provide non-limiting examples, 3 Fr (1 mm; 0.039 inches), 4 Fr (1.35 mm; 0.053 inches), 5 Fr (1.67 mm; 0.066 inches), 6 Fr (2 mm; 0.079 inches), 7 Fr (2.3 mm; 0.092 inches), and so on. The corresponding diameters in millimeters and inches are shown in parenthesis. The French system has uniform increments between gauge sizes (⅓ of a millimeter) (Iserson K V (1987) J.-F.-B. Charrière: the man behind the “French” gauge. J. Emerg. Med. 5:545-548). Systems for measuring the outside diameter and inside diameter (lumen) of catheters, needles, and the like have been described (see, e.g., Ahn, et al. (2002) Anesth. Analg. 95:1125). French size can refer to an inside diameter or to an outside diameter (see, e.g., U.S. Pat. No. 7,641,645 issued to Schur, which is hereby incorporated by reference).

### Copolymer Embodiments; Porosity Embodiments; Hydrogel Embodiments

Copolymers are encompassed by the disclosure, for example, copolymers of the block type and copolymers of the rake type (see, e.g., U.S. Pat. No. 8,008,407 of Oberhellman et al, and U.S. Pat. No. 8,084,535 of Maton et al, which are incorporated herein by reference in their entirety). Regarding porosity, if the porosity of a polymer coating is not sufficient to allow diffusion of an agent, such as a drug, into the extracellular fluids, a porosigen, such as lactose, can be added to the polymer used for the coating. Hydrogels, and methods for controlling water content of hydrogels, and mechanical strengths of various types of hydrogels are described (see, e.g., U.S. Pat. No. 4,734,097 of Tanabe et al, which is hereby incorporated by reference in its entirety). Because of their weak, rubbery mechanical properties, polysiloxane is sometimes prepared as chemically crosslinked, or synthesized as a block polymer that alternates with a harder type of polymer (see, page 36 of F. Wang (1998) Polydimethylsiloxane Modification of Segmented Thermoplastic Polyurethanes and Polyureas, Thesis, Virginia Polytechnic Institute and State Univ., Blacksburg, Va.).

By way of definition, an example of “one type” of plastic polymer is, for example, a polymer that comprises mainly polyurethane, mainly polysiloxane, mainly polyethylene, or mainly one type of copolymer. The skilled artisan will understand that modification of a polyurethane polymer with various end groups do not change the fact that the polymer is still classified as a type of “polyurethane.” A “copolymer” is defined as consisting mainly of “one type” of plastic polymer, because the two polymers in the copolymer are integrated together, and are also covalently bound to each other, for example, in the manner of a block copolymer or a rake copolymer.

### Couplers and Locks

In embodiments, the present disclosure provides a coupler or lock, which as Luer lock, or unisex Storz type coupler (see, e.g., U.S. Pat. No. 4,602,654 of Stehling et al). Locking tabs are provided (see, e.g., U.S. Pat. No. 5,885,217 issued to Gisselberg et al). Provided is coupler, where one or more radially-oriented protrusions fit into one or more radially-oriented grooves (see, e.g., U.S. Pat. No. 6,336,914 of Gillespie). Locking collar is encompassed (see, e.g., US 2005/0090779 of Osypka). Also provided is coupler, where proximal-to-distal (axially-oriented) pin or pins fit into one or more slots (see, e.g., US 2009/0143739 of Nardeo et al). Further provided, is threaded coupler (see, e.g., U.S. Pat. No. 7,422,571 of Schweikert et al). Each of the above patents and published patent applications are hereby incorporated herein by reference, in their entirety. In embodiments, what is encompassed is a valve, or a medical device that comprises a valve. A coupler can couple a first hub to a second hub, for example, a first hub that is a catheter hub and a second hub that is a needle hub. Or the first hub can be a catheter hub and the second hub can be a sheath hub. Valve of the present disclosure can reside in a housing that is a hub, or the valve can reside in a housing that is not a hub.

### Reduced Friction Embodiments; Plastic Memory Embodiments

Silicone can reduce the coefficient of friction. Methods for applying silicone and for measuring coefficient of friction are available (see, e.g., U.S. Pat. No. 5,013,717 of Solomon, which is hereby incorporated by reference in its entirety). What is provided is medical device that retain their “plastic memory,” such as medical device comprising thermoplastic polyurethane, as compared to vinyl resin (see, e.g., U.S. Pat. No. 4,579,879 of Flynn, which is hereby incorporated herein by reference in its entirety). What is provided is medical device that, in its entirety, or in segments, comprises siloxane. Medical device comprising siloxane has increased flexibility, when compared, for example, to a medical device that is substantially made of polyurethane (see, e.g., U.S. Pat. No. 8,092,522 of Paul et al, which is hereby incorporated by reference in its entirety).

Balloons, fabrics for balloons, layers, adhesives, housings for balloons, devices for inserting and withdrawing balloons, related devices such as stents and catheters, methods of manufacture, and methods for administration, treatment, or diagnosis, and methods for insertion or withdrawal of a medical device from a patient, are available. See, for example, U.S. Pat. No. 7,862,575 of Tal; US 2007/0060882 of Tal, US 2011/0160661 of Elton; US 2010/03180 of Pepper). Each of these patents and published patent applications is hereby incorporated by reference as if set forth herein in its entirety.

The hardness of the devices of the present disclosure, including hardness of specific features, such as a tip, wall, bump, tapered region, hub, wing, tab, conical region, bead-like region, can be measured by the durometer method and Shore hardness scale. See, e.g., U.S. Pat. No. 5,489,269 issued to Aldrich et al, U.S. Pat. No. 7,655,021 issued to Brasington et al, and Eleni, et al. (2011) Effects of outdoor weathering on facial prosthetic elastomers. Odontology. 99:68-76, which are each individually incorporated herein by reference in their entirety. Shore A hardness refers to hardness determined where a steel rod dents in the material, while Shore D hardness refers to hardness that is determined where a steel rod penetrates into the material. Shore hardness, using either the Shore A or Shore D scale, is used for rubbers/elastomers and is also commonly used for softer plastics such as polyolefins, fluoropolymers, and vinyls. The Shore A scale is used for softer rubbers while the Shore D scale is used for harder rubbers.

The viscosity of solutions and formulations, including those comprising polyurethane can be measured using available instruments and methods. See, for example U.S. Pat. No. 8,017,686 issued to Buter, et al, and U.S. Pat. No. 5,091,205 issued to Fan, which are hereby incorporated by reference. The Brookfield viscometer is a standard instrument (Brookfield Engineering Laboratories, Middleboro, Mass.). Equipment and methods for burst tests are available. See, e.g., Uson Testra static burst tester; Uson, Houston, Tex. The burst test can be destructive or non-destructive.

Thermoplastic polyurethane (TPU) tubing, resins, and the like, are available for use in the present disclosure, for example, as a medical device such as a catheter, as a coating for the medical device, as a formula configured for use in coating the medical device, or as a medical device that is modified by coating with the formula. What is available is tubing, resins, and the like, having a hardness of 72A, 77A, 87A, 94A, 51D, 60D, 63D, 67D, 73A/78A, 83A/86A, 90A/95A, 93A/98A, 55D/65D, 63D/78D, 73D, 75D/82D (Tecoflex® series); and 75A, 85A, 94A, 54D, 64D, 69D, 74D, 75D, 77A/83A, 87A/88A, 97A/97A, 55D/64D, 67D/75D, 70D, 75D, 77D/84D (Tecothane® series) (Lubrizol's Engineered Polymers for Medical and Health Care; Lubrizol Corp, Cleveland Ohio). Guidance on medical polymers, including polyurethane, is available, for example, from Polymer Membranes/Biomembranes (Advances in Polymer Science), ed. by Meier and Knoll, Springer, 2009; Lubricating Polymer Surfaces by Uyama, CRC Press, 1998; and Polymer Grafting and Crosslinking, ed. by Bhattacharya, et al, Wiley, 2008.

Reagents, including high purity solvents, as well as polymer resins such as 95A resin, can be acquired from Lubrizol Corp., Cleveland, Ohio; Microspec Corp., Peterborough, N.H.; Polaris Polymers, Avon Lake, Ohio; U.S. Plastic Corp., Lima, Ohio; Sigma-Aldrich, St. Louis, Mo.; E.I. du Pont de Nemours and Company, Wilmington, Del.; Dow Chemical Co., Midland, Mich. Polyurethane of durometer 95A is disclosed, for example, by US 2010/0082097 of Rosenblatt, et al, U.S. Pat. No. 6,517,548 issued to Lorentzen Cornelius, et al, and by US 2011/0054581 of Desai and Reddy. Each of these is hereby incorporated herein by reference.

Methods and equipment are available to the skilled artisan for measuring structures, properties, and functions, of medical devices, such as catheters. The following references disclose methods and equipment for measuring, for example, tensile strength, force at break, elastic behavior, plastic behavior, microscopy for detecting microbial colonies or biofilms residing on the surface of catheters, microbiological assays for measuring influence of anti-microbials. See, e.g., Aslam and Darouiche (2010) Infect. Control Hosp. Epidemiol. 31:1124-1129; Hachem et al (2009) Antimicrobial Agents Chemotherapy 53:5145-5149; Venkatesh et al (2009) J. Medical Microbiol. 58:936-944, which are hereby incorporated herein by reference. Methods and equipment for measuring tensile strength, elongation at break, and other properties of medical devices, are available. See, e.g., U.S. Pat. No. 6,039,755 issued to Edwin et al, and U.S. Pat. No. 7,803,395 issued to Datta et al, which are incorporated herein by reference. Above a limiting stress, called the elastic limit, some of the strain is permanent. In going beyond the elastic limit, a solid can either fracture suddenly or deform in a permanent way (see, e.g., Ashby M F, Jones D R H (2012) Engineering Materials 1, 4^{th} ed., Elsevier, New York, pp. 115-133).

### Coatings

The present disclosure provides, without limitation, coatings that comprise sulfobetaine, or carboxybetaine, hydrogels, polyurethane, polyester, polyethylene, polyamide, mixtures thereof, diblock polymers, layered coatings, interpenetrating polymer networks, See, e.g., U.S. Pat. No. 7,879,444 issued to Jiang et al; US 2009/0259015 of Jiang and Chen; US 2009/0155335 of O'Shaughnessey et al; US 2009/0156460 of Jiang et al; US 2010/0145286 of Zhang et al; 2011/0097277 of Jiang et al; and US 2010/0152708 of Li et al, each of which is individually incorporated herein by reference in its entirety.

### EXAMPLES

Linkers of the present disclosure encompass bi-functional linkers, where a first functional group reacts with a drug, and where this functional group can be called a “protecting group.” The “protecting group” can form for example, an ester. The second functional group can bond (hydrogen bond or covalent bond) to a polymer matrix, resulting in immobilization to the matrix. Bonding and immobilization can be via hydrogen bonds, one or more covalent bonds, or a combination of hydrogen bonds and covalent bonds. For example, a 3,5-dimethoxyenzoin (DMB) which is functionalized with a thiol can react with a drug to form an ester (through the DMB alcohol), and can react to a PVC surface through the thiol. The thiol and alcohol are in separate parts of the molecule. The PVC-immobilized molecule can then have the ester cleaved by light (equivalent to deprotection), liberating the drug. The second functional group can also be an alkenyl group.

In multi-functional embodiments, the linker can have two protecting groups, three, four, five, or more protecting groups. The protecting groups can be identical to each other, for example, they can each be propyl-aldehyde group. Or the protecting groups can differ from each other, that is, one can contain an aldehyde group, while the other can bear a sulfhydryl group. Moreover, the linker can have two or more second functional groups. Again, these can all have the same, identical structure, or they can be different in nature.

The present disclosure provides angioplasty balloon that is coated or impregnated with, or otherwise processed to include, an anti-restenosis agent. The anti-restenosis agent is configured so that, during plaque compaction by balloon inflation, the active coating is in contact with the vessel wall.

In embodiments, active agent can be a synthesized conjugate molecule that remains immobilized and inactive within the polymer coating, until activated by a specific spectrum and fluence level of photonic energy. Method of agent activation can be the photocleaving of one or more chemical bonds connecting the active agent to the immobilizing element of the conjugate molecule.

In one embodiment, the coating contains only one type of pharmaceutical agent. In other embodiments, the coating contains more than one type of pharmaceutical agent. In one embodiment, the coating contains only one type of photocleavable moiety. In another embodiment, the coating contains more than one type of photocleavable moiety.

The photo-cleaving energy can be delivered to the conjugate by direct delivery of the critical photo-cleaving spectrum by an optical fiber which has been processed to provide a specific region of side emission, equal in length to the coated length of the balloon.

In another non-limiting embodiment, delivery of a longer wavelength spectrum to a photo up-conversion material such as inorganic phosphor crystals. These crystals can be uniformly distributed or compounded into the polymer coating along with the photocleavable agent. The up-conversion crystals shall convert the longer wavelength energy, as transmitted by an optical fiber processed for side emission, to a shorter wavelength capable of photo-cleaving the conjugate molecule.

Transmission and delivery of the photo-cleaving energy can be triggered either manually by the clinician during inflation of the balloon and plaque compaction, or in an automated fashion by microprocessor measurement of the digitally transduced balloon pressure at a value (atmospheres of pressure) predetermined by the clinician to represent a fully inflated balloon.

In non-limiting embodiments, fiber transmission of the photocleaving energy can be accomplished by either: (1) Integration of the fiber optic element within an existing inflationary lumen of the hotspur catheter; or (2) Integration of the fiber optic element with the guide wire or stylet (hollow construction). Direct substitution of the guide wire or stylet by fiber optic element is an optional embodiment.

Optical energy transmitted by one or more fiber optic elements can originate from and be coupled to either a coherent (laser) or incoherent (light emitting diode; LED) source controlled by direct photonic measurement (PD feedback loop) by a SiC (Silicon carbide) photo detector and a microprocessor circuit.

The present disclosure provides devices, and relevant methods, with the ability perform multiple doses of drug delivery. Multiple does of drug deliver can be either within the same lesion or at another point in the vascular. The lesion can be an atherosclerotic lesion, a cancerous lesion, or another pathological structure. This may require sufficient loading of the drug up-front and optionally a secondary molecular bond and corresponding wavelength of light to trigger the additional release.

Preferred light-activated protecting groups are 3,5-dimethoxybenzoin derivatives and ortho-nitrobenzyl derivatives. The following concerns embodiments where the pharmaceutical agent is paclitaxel. For protection or deprotection of the alcohol of paclitaxel, a preferred derivative is o-nitrobenzyl derivatives.

While the method and apparatus have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

It should also be understood that a variety of changes may be made without departing from the essence of the invention. Such changes are also implicitly included in the description. They still fall within the scope of this invention. It should be understood that this disclosure is intended to yield a patent covering numerous aspects of the invention both independently and as an overall system and in both method and apparatus modes.

Further, each of the various elements of the invention and claims may also be achieved in a variety of manners. This disclosure should be understood to encompass each such variation, be it a variation of an embodiment of any apparatus embodiment, a method or process embodiment, or even merely a variation of any element of these.

Particularly, it should be understood that as the disclosure relates to elements of the invention, the words for each element may be expressed by equivalent apparatus terms or method terms—even if only the function or result is the same.

Such equivalent, broader, or even more generic terms should be considered to be encompassed in the description of each element or action. Such terms can be substituted where desired to make explicit the implicitly broad coverage to which this invention is entitled.

It should be understood that all actions may be expressed as a means for taking that action or as an element which causes that action.

Similarly, each physical element disclosed should be understood to encompass a disclosure of the action which that physical element facilitates.

Any patents, publications, or other references mentioned in this application for patent are hereby incorporated by reference.

Finally, all references listed in the Information Disclosure Statement or other information statement filed with the application are hereby appended and hereby incorporated by reference; however, as to each of the above, to the extent that such information or statements incorporated by reference might be considered inconsistent with the patenting of this/these invention(s), such statements are expressly not to be considered as made by the applicant.

In this regard it should be understood that for practical reasons and so as to avoid adding potentially hundreds of claims, the applicant has presented claims with initial dependencies only.

Support should be understood to exist to the degree required under new matter laws—including but not limited to United States Patent Law 35 USC 132 or other such laws—to permit the addition of any of the various dependencies or other elements presented under one independent claim or concept as dependencies or elements under any other independent claim or concept.

To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, the use of the transitional phrase “comprising” is used to maintain the “open-end” claims herein, according to traditional claim interpretation. Thus, unless the context requires otherwise, it should be understood that the term “compromise” or variations such as “comprises” or “comprising”, are intended to imply the inclusion of a stated element or step or group of elements or steps but not the exclusion of any other element or step or group of elements or steps.

Such terms should be interpreted in their most expansive forms so as to afford the applicant the broadest coverage legally permissible.

What is claimed is:

1. A medical device configured for delivering at least one reagent to an intraluminal location, or other location in the body, the medical device comprising a surface and a coating, wherein the coating is modified by a photosensitive linker that covalently binds the at least one reagent agent, the photosensitive linker comprising:
(a) a first functional group that covalently binds the pharmaceutical agent;
(b) a photosensitive moiety that is adapted to cleaved by exposure to light; and
(c) a second functional group that maintains contact with the coating;
wherein the coating on the medical device is configured to deliver and, with exposure to light, release the at least one reagent in an effective amount that contacts the location.

2. The medical device of claim 1, wherein the at least one reagent comprises a pharmaceutical agent, a diagnostic agent, a medicament, or a nutrient.

3. The medical device of claim 1, wherein the device is configured for delivering at least one medicament to an intraluminal location that comprises atherosclerotic plaque or a location that is at risk for restenosis.

4. The medical device of claim 1, wherein the device is configured for delivering at least pharmaceutical agent to an intraluminal location that comprises atherosclerotic plaque or a location at risk for restenosis.

5. The medical device of claim 1, wherein the location is tissue that is at risk for restenosis, and wherein the location has a history of at least once being treated for atherosclerotic plaque.

6. The medical device of claim 1, wherein the device is configured for delivering at least one medicament to an intraluminal location that comprises a blood clot or embolism.

7. The medical device of claim 1 that comprises at least one angioplasty balloon, a stent, and a vascular device, that is configured for temporary or permanent placement.

8. The medical device of claim 1, wherein the second functional group maintains contact with the coating by hydrogen bonds, and not by one or more covalent bonds.

9. The medical device of claim 1, wherein the second functional group maintains contact with the coating by at least one covalent bond.

10. The medical device of claim 1, wherein the at least one reagent is cytostatic.

11. The medical device of claim 1, wherein the at least one reagent is cytotoxic.

12. The medical device of claim 1, where the at least one reagent includes a taxol, or an analogue thereof.

13. The medical device of claim 1, wherein the at least one reagent agent is paclitaxel.

14. The medical device of claim 1, wherein the coating comprises at least one of a polyurethane or a polysiloxane.

15. The medical device of claim 1, wherein the photosensitive linker comprises one or both of, at least one double bond that links two carbon atoms, at least one azo linkage, or at least one peroxide linkage.

16. The medical device of claim 1, further comprising a photo up-conversion material.

17. The medical device of claim 1, further comprising a photo up-conversion material that comprises inorganic phosphor crystals.

18. The medical device of claim 1, further comprising a light-emitting diode that delivers a photocleavably effective light to the coating.

19. The medical device of claim 1, further comprising a second medical device having at least one optical fiber that delivers a photocleavably effective light to the coating.

20. The medical device of claim 19 that occurs as an integral unit with the second medical device.

21. A kit comprising the medical device of claim 1, and a second medical device having at least one optical fiber that delivers a photocleavably effective light to the coating.

22. A kit comprising the medical device of claim 1, wherein the medical device comprises an angioplasty balloon that is coated with at least one photocleavable reagent, and a second medical device having at least one optical fiber that delivers a photocleavably effective amount of light to the coating.

23. A method for delivering, or releasing, at least one reagent to an intraluminal location, or other location in the body, comprising positioning the medical device of claim 1 in said intraluminal location, or other location in the body, and irradiating the coating with a photocleavably effective amount of radiation.

24. The method of claim 23, wherein the intraluminal location is an atherosclerotic lesion, or a lesion at risk for restinosis, and wherein the coating comprises a photocleavable link that is linked to paclitaxel.

25. A method of manufacturing the medical device of claim 1, comprising attaching a photocleavable linkage and at least one reagent to a surface of a non-coated medical device, wherein the photocleavable linkage tethers the at least one reagent to the surface of the medical device.

26. A method for delivering, or releasing, at least one reagent to an intraluminal location, or other location in the body, comprising the step of positioning a medical device in a lumen;
wherein the medical device is configured for delivering at least one reagent to an intraluminal location, or other location in the body, the medical device comprising a surface and a coating, wherein the coating is modified by a photosensitive linker that covalently binds the at least one reagent, the photosensitive linker comprising:
(a) a first functional group that covalently binds the reagent;
(b) a photosensitive moiety that is adapted to cleaved by exposure to light; and
(c) a second functional group that maintains contact with the coating;
wherein the coating on the medical device is configured to deliver and, with exposure to light, release the at least one reagent in an effective amount that contacts the location; and,
further comprising the step of irradiating the coating with a photocleavably effective amount of radiation.

27. The method of claim 26, wherein the intraluminal location is an atherosclerotic lesion, or a location that is at risk for restenosis, and the reagent is an anti-proliferative agent.
